(19) United States
(12) Patent Application Publication
(10) Pub. No.: US 2021/0045657 A1
(43) Pub. Date: Feb. 18, 2021

(54) DEVICE AND METHOD FOR MEASURING RESPIRATORY AIR FLOW

(71) Applicant: SINGAPORE HEALTH SERVICES PTE LTD, Singapore (SG)

(72) Inventors: Biju THOMAS, Singapore (SG); Zhi Qian HEN, Singapore (SG); Dirk Frans DE KORNE, Singapore (SG)

(73) Assignee: Singapore Health Services Pte Ltd, Singapore (SG)

(21) Appl. No.: 16/977,415

(22) PCT Filed: Mar. 4, 2019
(86) PCT No.: PCT/SG2019/050121

§ 371 Date: Sep. 1, 2020

(30) Foreign Application Priority Data
Mar. 2, 2018 (SG) 10201801727U

(51) Int. Cl. A61B 5/087; A61B 5/00; G16H 40/67

(52) U.S. Cl. CPC A61B 5/0871; A61B 5/0878; A61B 2562/0271; G16H 40/67; A61B 5/0022

(57) ABSTRACT

A device and method for measuring respiratory air flow of a subject are provided. The device includes a hollow member having a proximal end, a distal end and a flow passage formed between the proximal and distal ends. The proximal end is configured to be received in a mouth of a subject. A flow sensor, such as thermal mass flow sensor, is disposed in the flow passage and configured to sense characteristics of an air flow in the flow passage. A processor is communicatively coupled to the flow sensor and configured to determine, based on an output from the flow sensor, whether the sensed characteristics of the air flow correspond to predetermined parameters.

### FIELD OF INVENTION

The present disclosure relates broadly, but not exclusively, to devices and methods for measuring respiratory air flow.

### BACKGROUND

Asthma is a very common condition that affects approximately 300 million people globally, with an estimated prevalence of 10-15% in children in Singapore. The World Health Organization (WHO) estimates that there are about 65 million people with moderate to severe Chronic Obstructive Pulmonary Disease (COPD). Most patients with respiratory diseases such as asthma and COPD need regular controller and/or reliever medications in the form of inhalers. Despite the presence of guideline-based standard treatment protocols and regular follow-ups as part of integrated asthma care programs, a substantial proportion of children continue to have suboptimal asthma control whilst on therapy. Poor asthma control is associated with significant interval asthma symptoms that adversely affect the quality of life in these children. Children with poorly controlled asthma are at risk of developing acute asthma exacerbations that may require frequent unscheduled physician/emergency visits and/or hospital admissions. In addition, acute asthma exacerbations may even be life threatening. Poor asthma control can lead to an accelerated decline in lung function and development of fixed airflow obstruction in the longer term. The healthcare and economic burden of poorly controlled asthma can be substantial for individuals and countries.

While there are many factors that may contribute to poor asthma control, suboptimal adherence to treatment is a common cause. An example of an asthma treatment includes regular asthma controller therapy, which in most patients includes inhaled steroids from a Metered Dose Inhaler (MDI) with a valved holding chamber (VHC), also known as a spacer, for use once or twice a day. Studies have shown that poor medication adherence and incorrect inhaler technique are common (ranging from 40-70%) in children with asthma. As asthma is a long-term chronic disease that has no cure currently, the objective of asthma treatment is to control it. It is found that children with asthma that is under control have higher medication adherence compared to those whose asthma is not under control.

The assessment of treatment adherence is part of a clinical encounter with asthma patients (adults and children) and forms a key piece of information based on which important treatment decisions are made by the clinicians. Clinicians may decide to step up, maintain or step down the treatment based on the clinical assessment of asthma control, as recommended by the clinical practice guidelines. The treatment decision to step up, maintain or step down the treatment is heavily influenced by the assessment of asthma control and the reported treatment adherence. Therefore, an incorrect assessment of treatment adherence may result in unwarranted step up of treatment by the physician and this can result in significant side effects, unnecessary medication burden and increased cost of care. On the other hand, it has been noted that self-reported adherence often overestimates true medication adherence and an inappropriate step down of treatment may lead to persistent poor asthma control and increased risk of asthma exacerbations.

The use of a MDI with a VHC (or spacer) is the most commonly used form of asthma controller therapy in the paediatric age group. Such a therapy is also recommended for a substantial proportion of adults with asthma or COPD. There are a number of VHCs available having variations in the size, shape and the materials used for making them. FIG. 1 shows a diagram 100 illustrating the steps in a general use of a MDI and a VHC by a patient. The method includes: at step 102, shaking the MDI device; at step 104, inserting the mouthpiece of the MDI into the rubber-sealed end of the spacer; at step 106, breathing all of the air out of the lungs, and placing the spacer into the mouth between the teeth so as to make a tight seal around the mouthpiece with the lips; at step 108, pressing the metered-dose inhaler down once to release the medicine. The medicine will be trapped in the spacer and the patient breathes in slowly and deeply. Finally, at step 110, the method includes holding the breath for 5 to 10 seconds before breathing out slowly. Alternatively, if the patient cannot hold his/her breath, the patient can breathe in and out slowly for 3 to 5 breaths.

However, errors in inhaler technique while executing the steps described above using the MDI with the VHC are common. These errors may include: the patient breathing in and out too fast (i.e. a panting type of breathing); the patient having a variable breathing pattern and/or the inspiratory flow rate is either too low or exceeds the recommended inspiratory flow rate (15-30 L/min); the patient has the MDI connected to the VHC with the mouth piece of the VHC in his/her mouth, but the patient breathes in and out through the nose (which means that he/she does not get any medication); the patient has the MDI connected to the VHC; with the mouth piece of the VHC in his/her mouth and the patient breathes in through the nose and then breathes out through the mouthpiece (meaning the patient will not get any medication).

The assessment of treatment adherence, e.g. asthma controller therapy by inhaling steroid using MDI with VHC, in routine clinical practice is typically based on patient's self-reporting, pharmacy records of prescriptions and inhaler check including checking the dose counter available on some inhalers. These tools have many limitations and treatment adherence is often over estimated. For example, the mere documentation of collection of medicines from pharmacy or dose counter/inhaler check does not confirm true medication adherence, i.e. whether the patient has been taking the medication correctly, as recommended by the physician. Hence, tools to assess adherence with asthma medications objectively are necessary.

In recent times, researchers have tried to develop tools to assess adherence with asthma medications and some examples of such tools include devices that act as dose counters (keeps track of number of left over doses), Smart Track devices (i.e. electronic device that captures data on the number of times the MDIs are actuated) and mobile phone applications. These tools/devices have limitations. In particular, the use of simple dose counters may not assist in the objective assessment of treatment adherence as they only indicate how many times the device was actuated. For example, a person could actuate the device as many times as needed to get the desired number of leftover doses, without inhaling any medication. Therefore, these devices do not give any evidence of the correct use of MDI through the VHC. Further, the patient may actuate the MDI correctly, but may use the inhaler using a direct method (i.e. without the VHC), inhale the medication using the VHC using an incorrect technique or may not inhale the medication at all (i.e. actuate the MDI only to get the desired count on the dose counter). There is also the potential issue of ‘contrivance’, which is the case where the patient may demonstrate the correct inhaler technique using the VHC at clinic reviews, but may voluntarily choose to use an alternate suboptimal technique in the home setting.

A need therefore exists to provide a device and a method for measuring inhalation technique and adherence to medications (in patients on treatment with pressurised metered dose inhaler (pMDI) with spacer) that seeks to address at least some of the above problems and limitations.

### SUMMARY

An aspect of the present disclosure provides a device for measuring respiratory air flow of a subject. The device comprises a hollow member having a proximal end, a distal end and a flow passage formed between the proximal and distal ends, wherein the proximal end is configured to be received in a mouth of a subject. A flow sensor is disposed in the flow passage and configured to sense characteristics of an air flow in the flow passage. A processor is communicatively coupled to the flow sensor and configured to determine, based on an output from the flow sensor, whether the sensed characteristics of the air flow correspond to predetermined parameters.

Another aspect of the present disclosure provides a method for measuring respiratory air flow of a subject. The method comprises the steps of inserting a proximal end of a hollow channel into a mouth of the subject; sensing, by a flow sensor, characteristics of an air flow in a flow passage formed between the proximal end and a distal end of the hollow member; and determining, by a processor, based on an output from the flow sensor, whether the sensed characteristics of the air flow correspond to predetermined parameters

### BRIEF DESCRIPTION OF THE DRAWINGS

The accompanying Figures, where like reference numerals refer to identical or functionally similar elements throughout the separate views and which together with the detailed description below are incorporated in and form part of the specification, serve to illustrate various embodiments and to explain various principles and advantages in accordance with a present embodiment, by way of non-limiting example only.

Embodiments of the invention are described hereinafter with reference to the following drawings, in which:

FIG. 1 shows a diagram illustrating the steps in a general use of a pressurised metered dose inhaler (pMDI) and a valved holding chamber (VHC).

FIGS. 2A to 2C show a perspective view, a plan view and a front view of a device for measuring respiratory air flow respectively, according to an example embodiment.

FIG. 2D shows a schematic layout of a circuit housing of the device of FIG. 2A, according to an example embodiment.

FIG. 3 shows a cross sectional view of the device of FIG. 2A when used with a pressurised metered dose inhaler (pMDI) and a valved holding chamber (VHC), according to an example embodiment.

FIG. 4 shows a schematic diagram of an operational amplifier differential amplifier circuit of the device of FIG. 2A, according to an example embodiment.

FIG. 5 shows a schematic diagram illustrating the transmission of data stored in the device of FIG. 2A, according to an example embodiment.

FIGS. 6A and 6B show schematic diagrams illustrating user interfaces of a mobile application installed in a mobile device of FIG. 5, according to an example embodiment.

FIG. 7 shows a graph illustrating a breathing pattern of a subject using the device of FIG. 2A, according to an example embodiment.

FIGS. 8A to 8J show graphs illustrating breathing patterns of various subjects in a study using the device of FIG. 2A, according to an example embodiment. In each figure, Graph A represents the baseline inhaler technique of the subject and Graph B represents the improved inhalation technique following individualized feedback to the subject

FIG. 9 shows a flow chart illustrating a method for measuring respiratory air flow rate, according to the example embodiments.

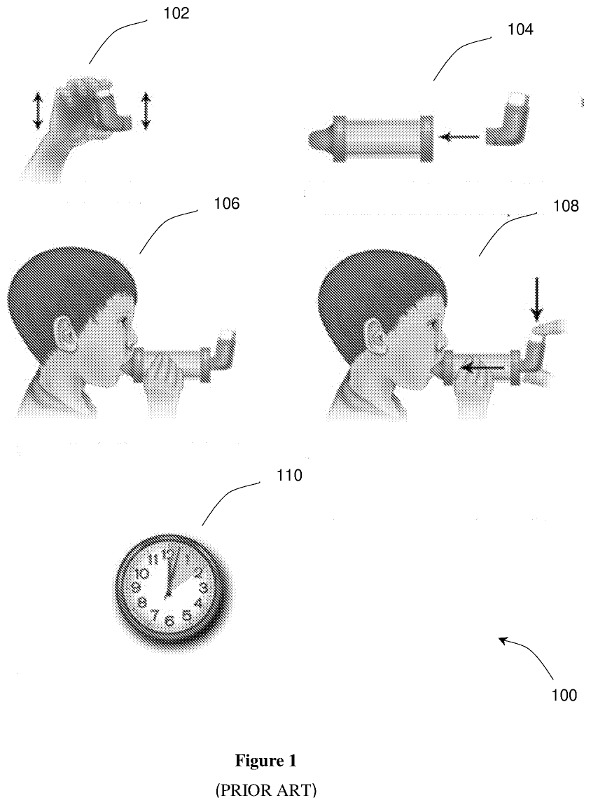

### DETAILED DESCRIPTION

The following detailed description is merely exemplary in nature and is not intended to limit the invention or the application and uses of the invention. Furthermore, there is no intention to be bound by any theory presented in the preceding background of the invention or the following detailed description. Herein, a device and a method for measuring respiratory air flow are presented in accordance with present embodiments having the advantages of having an accurate and objective monitoring of medication adherence (pressurised Metered Dose Inhaler (pMDI) and spacer) and inhaler technique in patients with respiratory diseases.

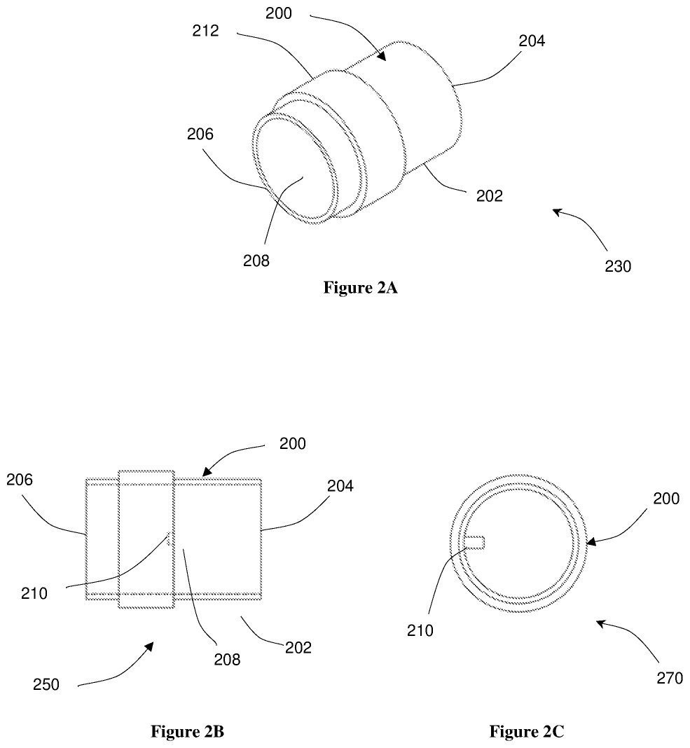

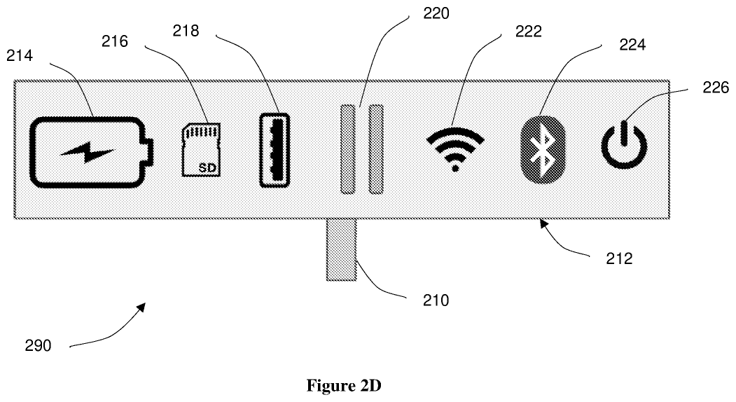

FIGS. 2A to 2C show a perspective view 230, a plan view 250 and a front view 270, respectively, of a device 200 for measuring respiratory flow according to an example embodiment. The device 200 includes a hollow member 202 having a proximal end 204, a distal end 206 and a flow passage 208 formed between the proximal and distal ends. The proximal end 204 is configured to be received in a mouth of a subject. A flow sensor 210 is disposed in the flow passage 208 and is configured to sense characteristics of an air flow in the flow passage 208. As an example, the flow sensor 210 may be a thermal mass flow sensor sensing air flow characteristics and may be integrated with the hollow member 202 such that they form a single unit. Some examples of the characteristics of the air flow include, but are not limited to, temperature, flow rate, and flow direction. The flow sensor 210 may also be a separate unit from the hollow member 202 and can be removably attached to the hollow member 202 using conventional attaching means. In alternative embodiments, the distal end 206 of the device 200 may also be received in the mouth of the subject.

The device 200 also includes a processor (not shown in FIGS. 2A-2C) communicatively coupled to the flow sensor 210 and configured to determine, based on an output from the flow sensor 210, whether the sensed characteristics of the air flow correspond to predetermined parameters. The processor may be integrated or separate from the hollow member 202. In example embodiments, the processor and the flow sensor 210 may form a single unit that can be removably attached to the hollow member 202 using conventional attaching means.

FIG. 2D shows a schematic layout 290 of a circuit housing 212 of the device 200 of FIG. 2A, according to an example embodiment. The circuit housing 212 may include the flow sensor 210 and other components such as a battery 214, a solid state storage drive 216 (e.g. micro-SD card), a USB port 218, LED indicator lights 220, WIFI components 222, Bluetooth components 224 and a power button 226. The circuit housing 212 may also include the processor described above. The circuit housing 212 together with the components is typically attached to the hollow member as shown in FIG. 2A.

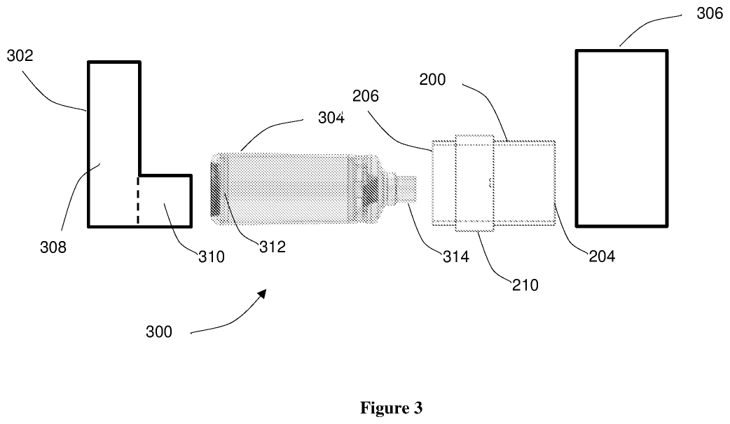

FIG. 3 shows a cross sectional view 300 of the device 200 of FIG. 2A when used with a pressurised metered dose inhaler (pMDI) 302 and a valved holding chamber (VHC or spacer) 304, according to an example embodiment. In this example, the distal end 206 of the device 200 is configured to be attached to the VHC 304 which in turn is attached to the pMDI 302. In addition, the proximal end 204 of the device 200 is configured to be received by the mouth of a subject 306. The device 200 can be used with any type of commercially available VHC.

In alternate embodiments, the distal end 206 of the device 200 may be placed at the mouth of the subject 306 and the proximal end 204 may be attached to the opposite tapered end 314 of the VHC 304. In another embodiment, the distal end 206 of the device 200 may be attached directly to the end 310 of the pMDI 302 without using the VHC 304. Furthermore, the distal end 206 of the device 200 may be configured in such a way that it can be attached directly to the mouth piece of asthma drug delivery devices (also known as inhalers) other than pMDI (such as dry powder inhalers, accuhaler, turbohaler, etc.). In other words, the device 200 is versatile and compatible with different devices without any physical modification.

In the example shown in FIG. 3, the pMDI 302 may include a medication portion 308 containing the medication for the subject 306 and a spacer end 310 configured to be attached to the VHC 304. The medication may be in any form which can be aerosolized. During use of the device 200, the pMDI 302 is first shaken. The spacer end 310 of the pMDI 302 is attached to one end 312 of the VHC 304 while an opposite tapered end 314 of the VHC 304 is attached to the distal end 206 of the device 200. Subsequently, the subject 306 exhales air out of his lungs and place his mouth on the proximal end 204 of the device 200.

The subject 306 then releases the medication contained in the pMDI 302. The medication release can be carried out by pressing a compressible member of the pMDI 302 or by activating a release catch in the pMDI 302. This will release the medicine into the VHC 304. The subject 306 then inhales which draws the medication in the VHC 304 through the device 200 into the mouth of the subject 306. After inhaling the medication, the subject 306 may hold his breath and exhale slowly. Alternately, the subject may breathe slowly and deeply in and out through the device 200 3-8 times so as to fully inhale the medication held in the VHC 304. In an alternate embodiment, the pMDI 302 may be directly attached to the device 200 without the VHC 304. The VHC 304 may include a one way valve mechanism that allows flow of the aerosolized medicine in one direction (i.e. towards the subject 306) when the subject 306 inhales. The exhaled air from the subject 306 will pass through the device 200 into end 314 of the VHC 304, and will be released to outside through the exhalation ports in the VHC 304, without entering the hollow chamber of VHC 304.

As the medication flows through the flow passage 208 of the device 200 when triggered by the subject's inspiratory effort, the flow sensor 210 detects air flow through the flow passage 208. The flow sensor 210 senses characteristics of the air flow which may include the quantity of the aerosolised medication and/or flow patterns. Other air flow characteristics that may be sensed by the flow sensor 210 include peak expiratory flow rate (PEFR), peak inspiratory flow rate (PIFR), maximum expiratory flow rate (MEFR) and maximum inspiratory flow rate (MIFR). The PIFR, PEFR, MIFR and the MEFR may be measured when the subject 306 exhales though the device 200 after a maximum inhalation, with the pMDI 302 and VHC 304 being disconnected from the device 200.

Measurement of the peak expiratory flow rate (PEFR) can be used in the diagnosis and management of subjects (or patients) with asthma. For example, during asthma diagnosis, day to day variability of PEFR readings may provide additional information along with clinical assessment. In patients with established diagnosis of asthma, a drop in PEFR readings from their predicted or personal best PEFR may help predict asthma exacerbations and/or in the assessment of the severity of asthma exacerbations.

A subject's PIFR information may be useful in deciding if the subject is able to use certain types of inhaler devices (such as the dry powder inhalers) properly. If the subject is unable to generate sufficient peak inspiratory flow rate (PIFR), certain types of inhaler devices will not be suitable for the patient.

The subject's respiratory muscle strength can be determined from the obtained MIFR and MEFR readings. More specifically, the MIFR and MEFR of the subject may be an indicator of the subject's maximum inspiratory/expiratory muscle strength, similar to measurements of Maximal Inspiratory Pressure (MIP) and Maximal Expiratory Pressure (MEP). The assessment of respiratory muscle strength may be useful in the management of children with neuromuscular diseases.

After the flow sensor 210 senses the air flow characteristics, it generates an output to the processor. Based on the output, the processor may determine whether the subject has inhaled the aerosolised medication, e.g. based on an inspiratory flow rate of an air flow containing the medication. The processor may also compare the sensed flow patterns with stored flow patterns, such as a desirable flow pattern or a target, and may also compare the sensed PEFR and PIFR with stored PEFR and PIFR. The processor may also compare the sensed MEFR and MIFR with stored MEFR and MIFR. The stored values of the flow patterns, PEFR, PIFR, MEFR, MIFR, etc. in the example embodiments may be historical data from the same subject, and can be useful in analysing trends and behaviours over time. Alternatively or in addition, the stored values can be preferred values or targets to help to train the subject for improvement or correction of inhalation technique.

By sensing characteristics of the air flow pattern, the device 200 may provide detailed information on the subject's inhaler technique such as inspiratory flow rate, breathing rate, breath holding pause, pause between breathes, average time taken for inhalation etc. This would help clinicians to review the subject's inhaler technique and provide focused, targeted and individualized inhaler technique education, using visual cues. Such feedback using visual cues may significantly improve the subject's inhaler technique so that drug delivery into lungs may be optimised.

The device 200 may include an indicator configured to display an indication whether the sensed characteristics of the air flow correspond to predetermined parameters. The indicator may be in the form of the indicator lights 220 of the housing circuit 212 of FIG. 2D and may provide an immediate indication to the subject 306 on his/her inhaler technique and/or quantity of medication inhaled. For example, the subject 306 (or patient) needs to inhale 10 millilitres of medication through the pMDI 302. When the subject 306 inhales, the flow sensor 210 senses the quantity of medication that is inhaled is only 3 millilitres and outputs the quantity to the processor. In this case, the processor compares the sensed quantity (3 millilitres) with the required quantity (10 millilitres) and sends a signal to the indicator (e.g. indicator lights 220) to light up as “red”, indicating that the subject 306 has not inhaled the required amount of medication. On the other hand, if the flow sensor 210 senses and outputs the quantity of medication as 10 millilitres, the processor compares and determines that the quantity is sufficient. A positive signal is then sent to the indicator (e.g. indicator lights 220) such that the indicator lights up as “green”, indicating the required amount of medication has been inhaled. It will be appreciated that the predetermined parameters may also be provided in ranges instead of absolute values. For example, if the inspiratory flow rate is within a pre-specified optimal range, the green indicator will light up, and if the inspiratory flow rate is outside the pre-specified optimal range (either lower of higher), the red indicator will light up. In alternate embodiments, the indicator display may be age and context specific, such as a rgamified' version where the user is nudged towards compliance with proper inhaler technique (e.g. earning points for correct inhalation).

The device 200 may further include a storage module communicatively coupled to the processor and configured to store the output from the flow sensor 210. In a preferred embodiment, the storage module may be in the form of the solid stage drive 216 as shown in FIG. 2D. In alternate embodiments, the storage module may include but is not limited to a magnetic tape, a miniature hard disk drive, a Read-Only Memory (ROM) or integrated circuit, a USB flash drive, a flash memory device, a solid state drive or a memory card), a hybrid drive, a magneto-optical disk, or a computer readable card such as a SD card and the like.

The data from the sensed characteristics of the air flow can be stored in the storage module (e.g. solid stage drive 216) and later downloaded and analysed (e.g. in an outpatient clinic setting) to assess the medication adherence and inhaler technique objectively. This feature allows capturing robust and accurate data on true medication adherence and inhaler technique using the device 200. For example, variabilities in breathing patterns while using the pMDI and the VHC may result in characteristic air flow patterns which can be used to review the inhaler technique. This can provide focused, targeted and individualised inhaler technique education using visual cues at a later stage. For example, the output from the sensor can be processed to derive a graphic representation of the air flow pattern which can be displayed for ease of understanding of the patient as well as the health care providers. Alternatively or in addition, real-time analysis can be done to provide the user with instant feedback about compliance with proper inhaler technique for confirmation or education.

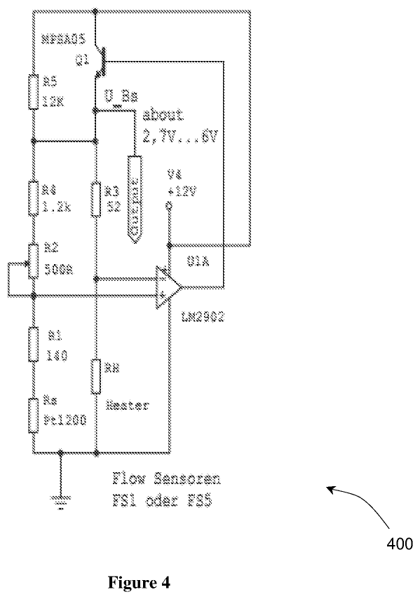

The device 200 may further include an operational-amplifier differential amplifier circuit. FIG. 4 shows a schematic diagram of an operational-amplifier (op-amp) differential amplifier circuit 400 of the device 200 of FIG. 2A, according to an example embodiment. The op-amp circuit 400 may be a bridging circuit with the flow sensor 210 and may be housed with the processor of the device 200 in the circuit housing 212 of the device 200 as shown in FIG. 2D.

The circuit housing 212 may be integrated with the hollow member 202 such that they form a single unit. In alternative embodiments, the circuit housing 212 may be a separate unit from the hollow member 202 and can be removably attached to the hollow member 202 using conventional attaching means. The flow sensor 210 may function on the principle of “hot element technique”, i.e. using temperature change to alter voltage levels. A Constant Temperature Anemometer (CTA) feedback circuit may be built together with the flow sensor 210 and by using King's Law, a graph showing the relation of temperature to flow velocity may be drawn and displayed.

The device 200 may also include a transmission module communicatively coupled to the processor and configured to transmit the output from the flow sensor to a remote device. The transmission module may be in the form of WiFi components 222 and/or the Bluetooth components 224 shown in FIG. 2D. The data stored in the storage module may be transmitted remotely via web-based resources or via mobile applications to a respiratory specialist. The stored data may be transmitted to the respiratory specialist or retrieved by the reviewing respiratory specialist during routine outpatient clinic review. This may provide information necessary for a tele-consultation with the respiratory specialist. This may also be helpful in tele-monitoring or virtual clinics as part of restructuring care pathways that involve application of telemedicine, thereby offering a potential for care transformation.

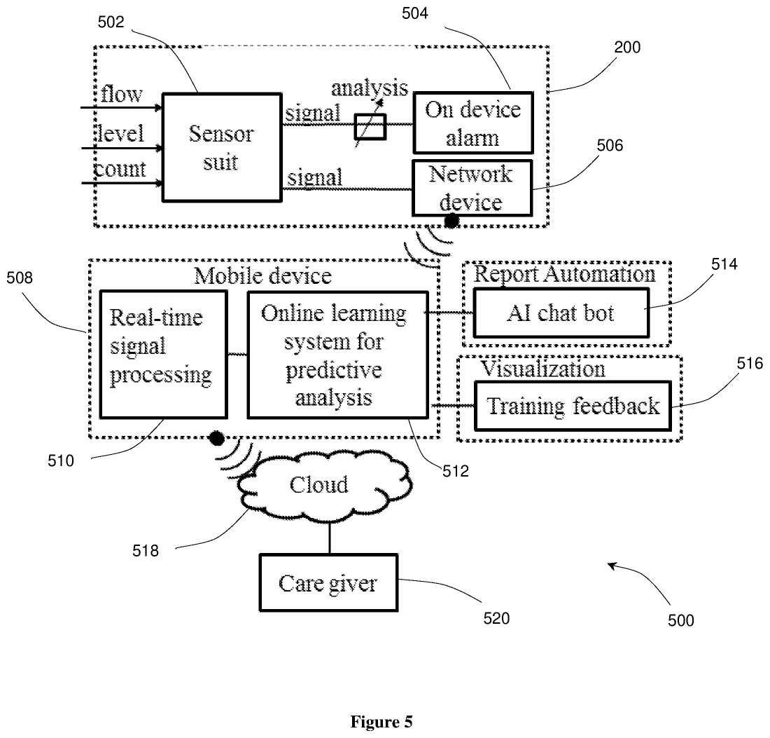

FIG. 5 shows a schematic diagram 500 illustrating the transmission of data stored in the device 200 of FIG. 2A, according to an example embodiment. In the Figure, the device 200 includes a sensor unit 502. The sensor unit 502 may include the housing circuit which may store the flow sensor 210, the op-amp circuit and the processor, where air flow characteristics are obtained and analyzed. The sensor unit 502 then relays a signal (or output) to the On device alarm 504, i.e. the indicator as described above, to display an indication whether the sensed characteristics of the air flow correspond to predetermined parameters. The sensor unit 502 may subsequently relay a transmission signal to a network device 506, i.e. the transmission module as described above. The transmission signal may include data of the sensed air flow characteristics from the sensor unit 502.

The network device 506 (or transmission module) may then transmit the signal and data from the device 200 to a mobile device 508. The mobile device 508 may include a real-time signal processing module 510 and an online learning system for predictive analysis module 512. The transmitted signal may then be further analyzed by the real-time signal processing module 510 and the online learning system for predictive analysis module 512. For example, the module 512 may be configured to detect patterns based on historical data, apply a look-up table, classification algorithm, machine-learning, etc. to make sense of the received data. The analyzed data may be transmitted to an Al chat bot 514 and a training feedback module 516 for reporting of the analyzed data to the subject 306. The Al chat bot 514 and the training feedback module 516 may be part of a mobile application that is installed in the mobile device 508. In such an implementation, an interactive system can be created where feedback or report can be provided automatically and in natural language. The analyzed data may also be transmitted to a cloud server 518 which a caregiver (or respiratory specialist) 520 has access and thus able to obtain the analyzed data and provide individualised feedback to the subject 306 to correct their inhaler technique. In some embodiments, the caregiver 520 may be able to access the data in real-time.

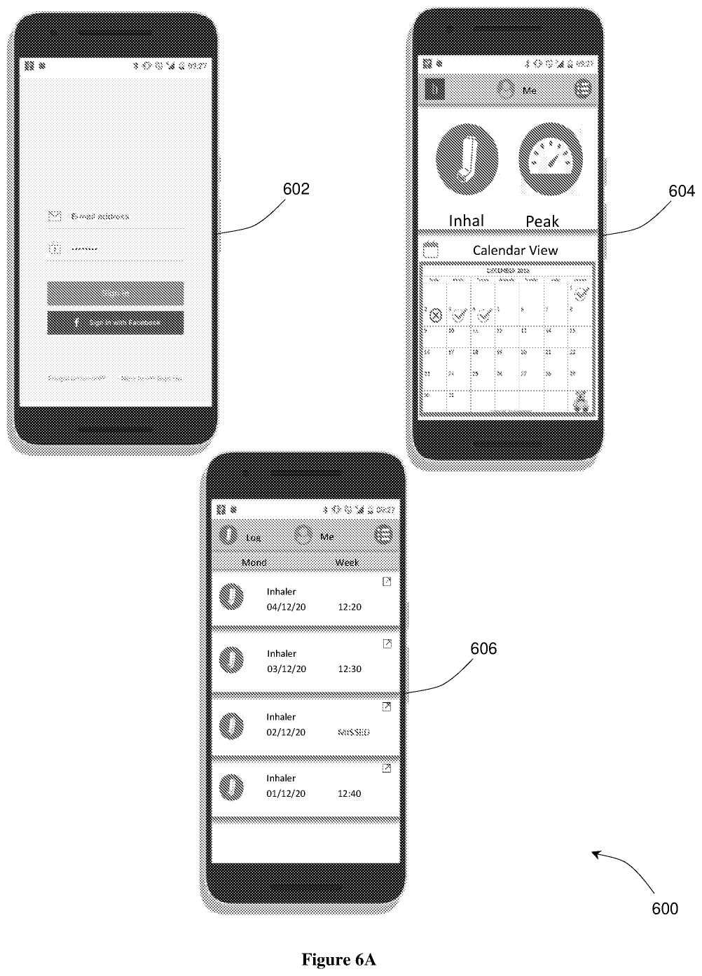

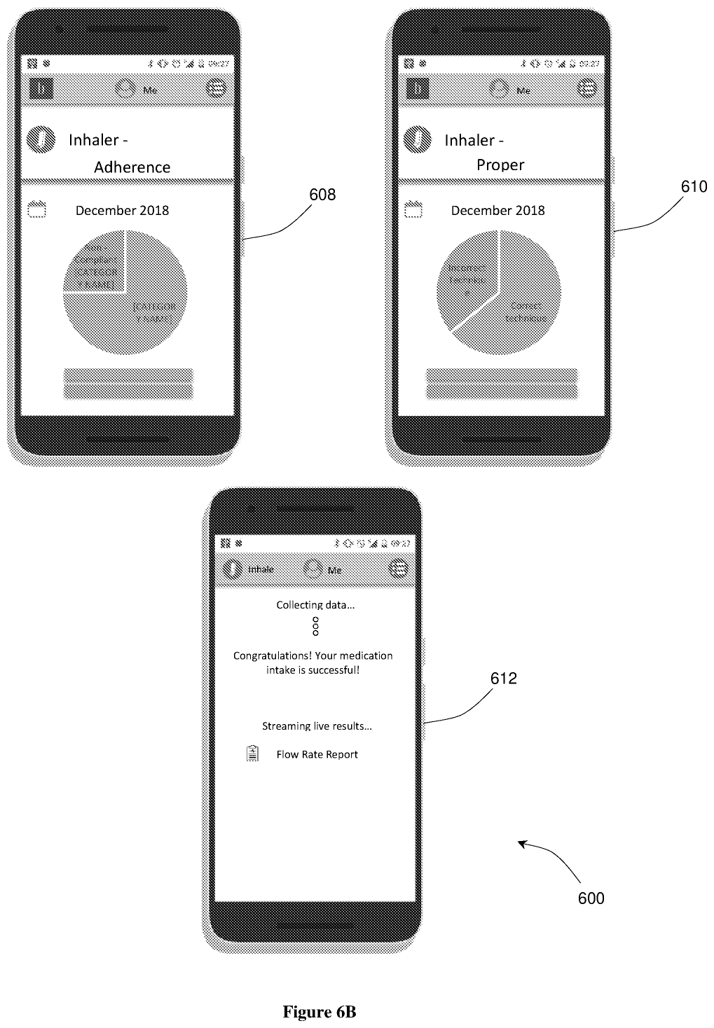

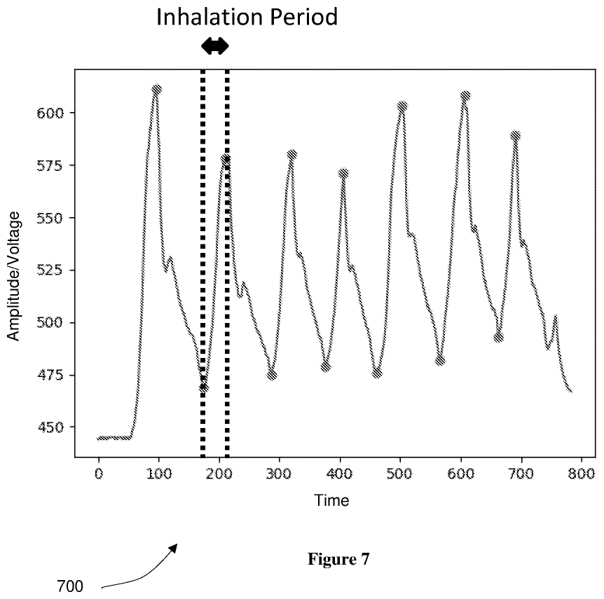

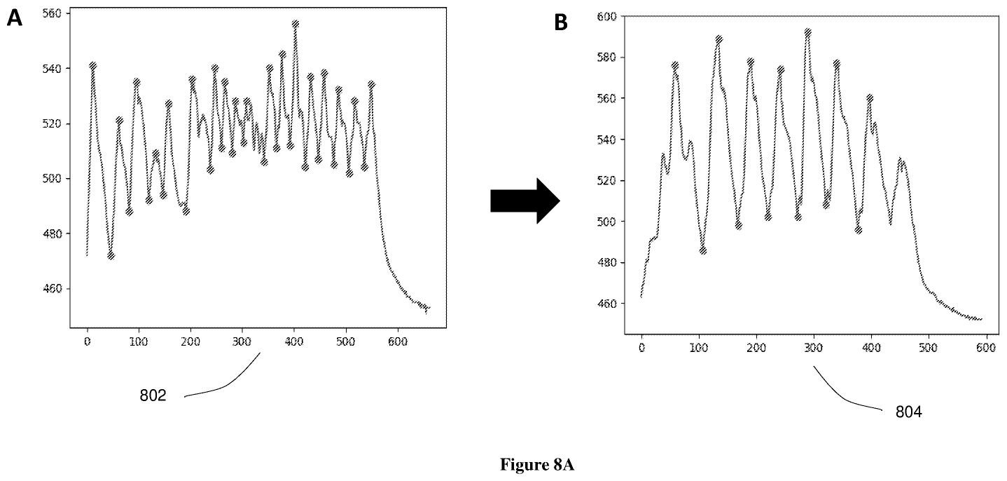

FIGS. 6A and 6B show schematic diagrams 600 illustrating user interfaces of a mobile application installed in a mobile device 508 of FIG. 5, according to an example embodiment. In FIG. 6A, the mobile application includes a login application interface 602 where the subject 306 may register and log in. Thereafter, the subject 306 may view his inhalation and medication data through a calendar interface 604 of the mobile application. The subject 306 may also view his medication data using a detailed view interface 606 where he is able to view the date and time of medication and also whether the medication has been successfully used.

As shown in FIG. 6B, the mobile application may also include an inhaler adherence interface 608 showing the subject's adherence to the inhaling technique per month via a pie chart or similar graphic representations. A proper inhaler interface 610 may be included in the mobile application showing a comparison of having a correct technique and an incorrect technique in a month through a pie chart or similar graphic representations. The mobile application can also collect data in real time when the subject 306 is using the device 200 to inhale his medication. This data can be displayed to the subject 306 using a “live” result interface 612 of the mobile application.

The mobile application may also be linked to the device 200 and compare the subject's PEFR reading and provide feedback, for example by displaying how the actual reading compares to the subject's predicted/personal best PEFR reading. A reading that falls below predetermined parameters may indicate the presence and severity of asthma exacerbations. The mobile application can be designed as a form of game for children or it may be a web based application that is easily accessible to subjects in order to facilitate active patient involvement in adherence and inhaler technique monitoring.

FIG. 7 shows a graph 700 illustrating a breathing pattern of a subject using the device 200 of FIG. 2A, according to an example embodiment. The graph 700, plotted using amplitude/voltage (y-axis) against time (x-axis), shows an optimal respiratory technique having an optimal inhalation period.

A study was conducted using an initial prototype of the device 200. The clinical study was conducted to test the device 200 in children with asthma in routine clinical context. 294 sets of data were collected on 49 children with asthma who are currently being followed up in specialist asthma clinics at a children hospital. The 49 children were aged between 6 to 18 years old and diagnosed with asthma. The study was conducted on each subject using three baseline measurements of their inhaler technique. Individualized feedback was provided to each subject using visual cues based on their captured flow pattern in order to correct their inhaler technique. This was followed by three measurements after post inhaler technique counselling of their inhaler technique. Hence, each subject has had six measurements giving a total of 294 data sets.

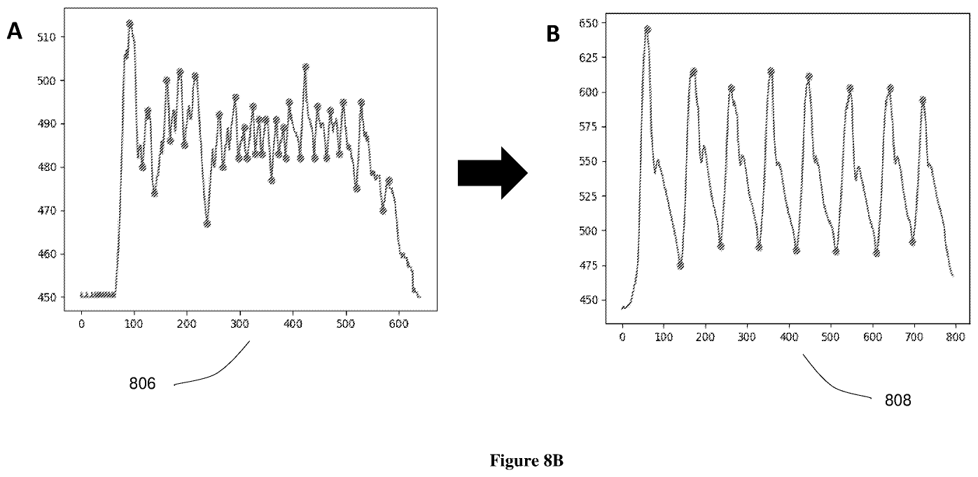

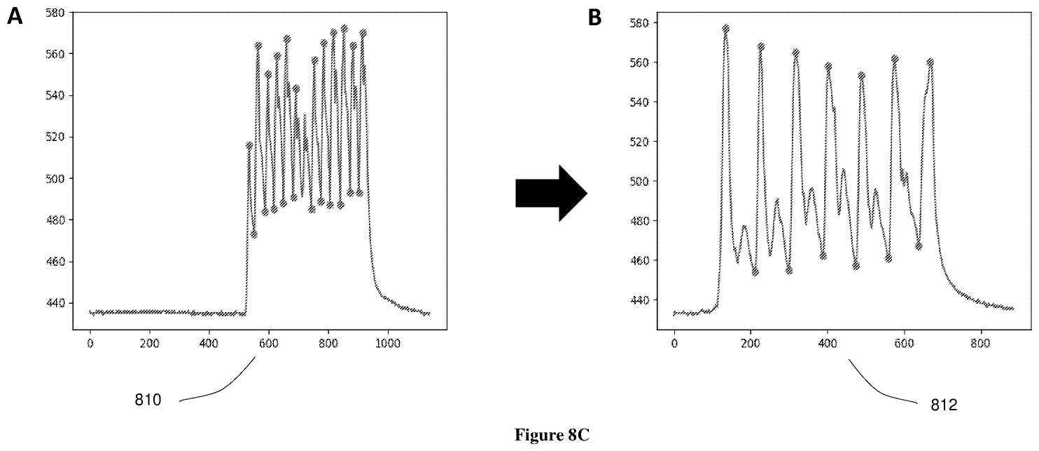

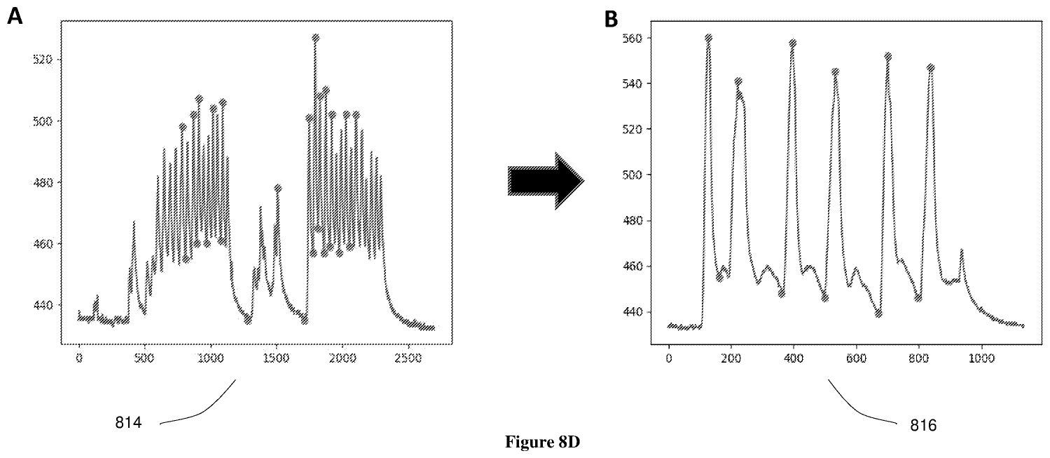

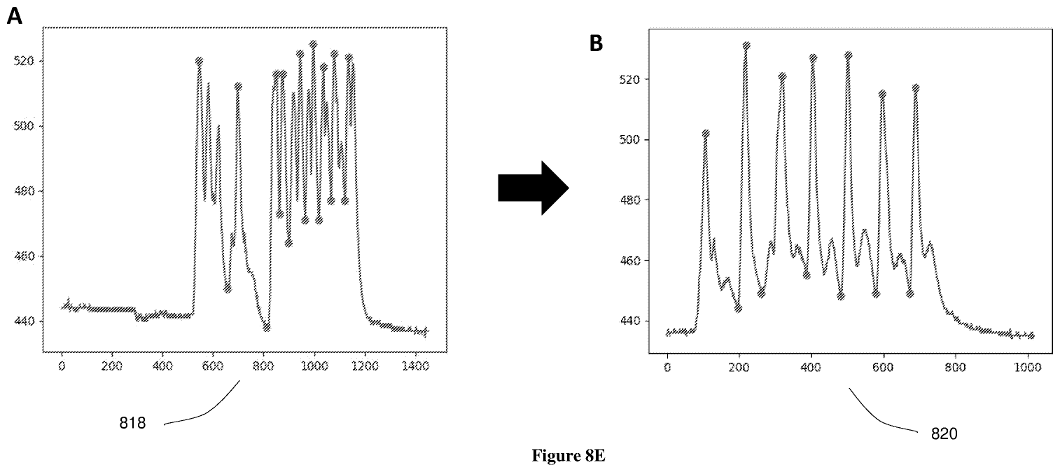

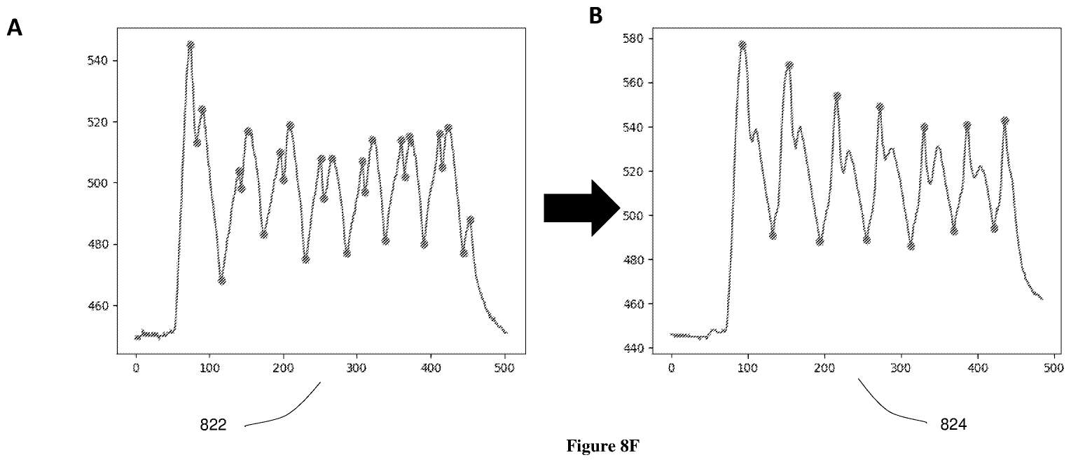

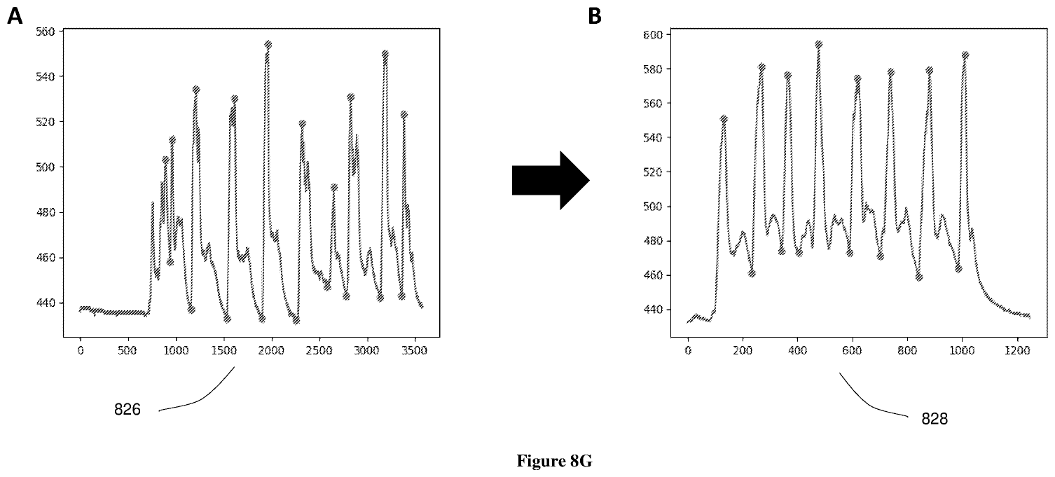

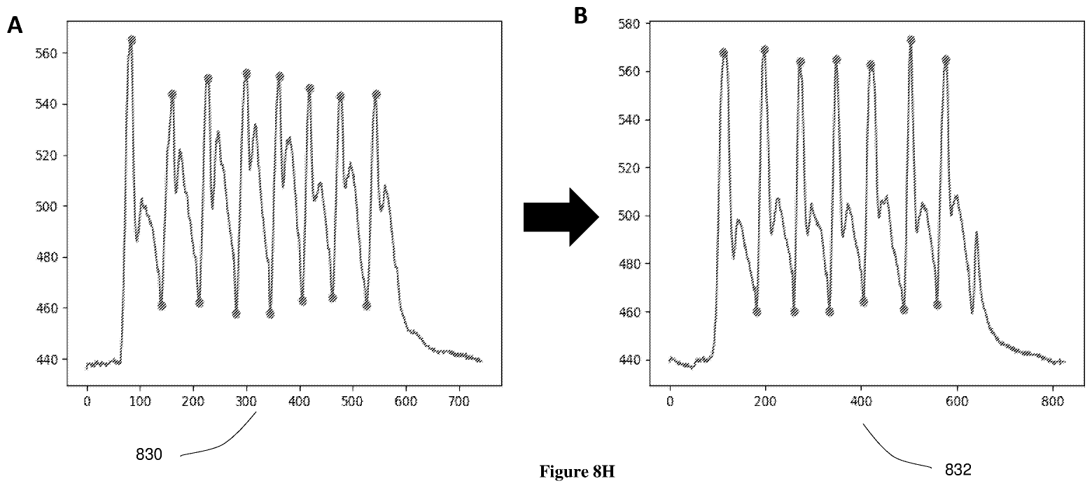

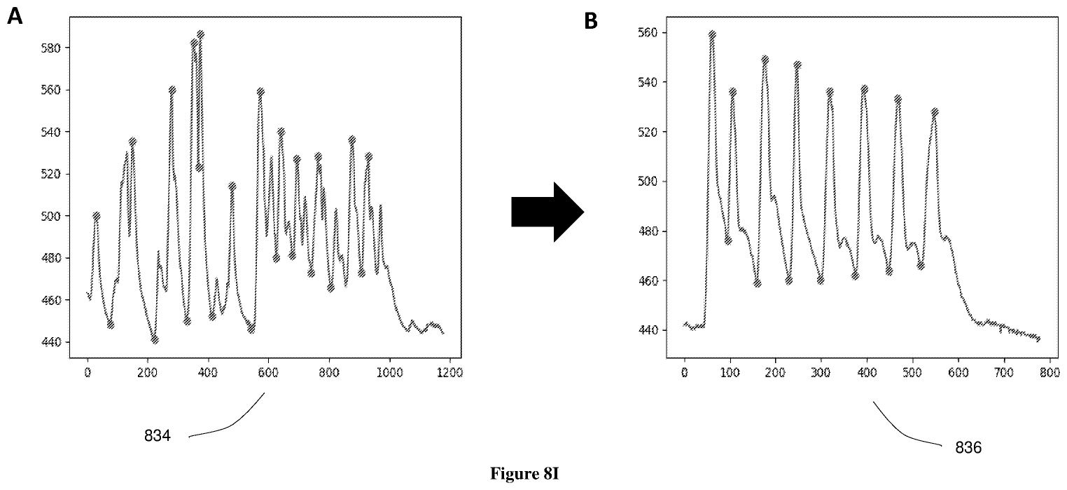

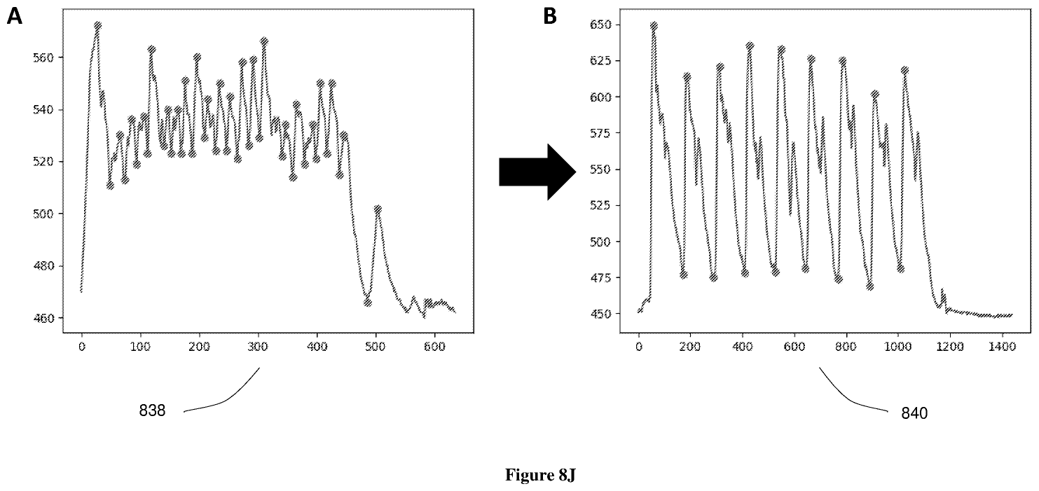

FIGS. 8A to 8J show graphs illustrating breathing patterns of various subjects in a study using the device of FIG. 2A, according to an example embodiment. In FIG. 8A, the data is presented from a 10 year old child using the device 200 with the pMDI 302 and VHC 304. The breathing pattern 802 (Graph A in FIG. 8A) of the child (i.e. subject) was captured and analyzed. Subsequently, individualized feedback to the child was made and an improved breathing pattern 804 (Graph B in FIG. 8A) was captured using the device 200. The improved breathing pattern 804 corresponds closely to the optimal respiratory technique as shown in FIG. 7.

In FIG. 8B, the data is presented from a 9 year old child using the device 200 with the pMDI 302 and VHC 304. The breathing pattern 806 (Graph A in FIG. 8B) of the child (i.e. subject) was captured and analyzed using the device 200 showing a rapid shallow breathing technique with insufficient inspiratory flow rate. Subsequently, individualized feedback using visual cues to the child was made and an improved breathing pattern 808 (Graph B in FIG. 8B) corresponding closely to the optimal respiratory technique as shown in FIG. 7 was captured using the device 200.

In FIG. 8C, the data is presented from a 8 year old child using the device 200 with the pMDI 302 and VHC 304. The breathing pattern 810 (Graph A in FIG. 8C) of the child (i.e. subject) was captured and analyzed using the device 200 showing an extremely fast panting type breathing technique. Subsequently, individualized feedback using visual cues to the child was made and an improved breathing pattern 812 (Graph B in FIG. 8C) corresponding closely to the optimal respiratory technique as shown in FIG. 7 was captured using the device 200.

In FIG. 8D, the data is presented from a 9 year old child using the device 200 with the pMDI 302 and VHC 304. The breathing pattern 814 (Graph A in FIG. 8D) of the child (i.e. subject) was captured and analyzed using the device 200 showing an extremely fast panting type breathing technique similar to FIG. 8C. Subsequently, individualized feedback using visual cues to the child was made and an improved breathing pattern 816 (Graph B in FIG. 8D) corresponding closely to the optimal respiratory technique as shown in FIG. 7 was captured using the device 200.

In FIG. 8E, the data is presented from a 7 year old child using the device 200 with the pMDI 302 and VHC 304. The breathing pattern 818 (Graph A in FIG. 8E) of the child (i.e. subject) was captured and analyzed using the device 200 showing a variable, fast breathing technique. Subsequently, individualized feedback using visual cues to the child was made and an improved breathing pattern 820 (Graph B in FIG. 8E) corresponding closely to the optimal respiratory technique as shown in FIG. 7 was captured using the device 200.

In FIG. 8F, the data is presented from a 14 year old child using the device 200 with the pMDI 302 and VHC 304. The breathing pattern 822 (Graph A in FIG. 8F) of the child (i.e. subject) was captured and analyzed using the device 200 showing an inhalation technique with short inspiratory pauses followed by forcible exhalation. Subsequently, individualized feedback using visual cues to the child was made and an improved breathing pattern 824 (Graph B in FIG. 8F) corresponding closely to the optimal respiratory technique as shown in FIG. 7 was captured using the device 200.

In FIG. 8G, the data is presented from a 8 year old child using the device 200 with the pMDI 302 and VHC 304. The breathing pattern 826 (Graph A in FIG. 8G) of the child (i.e. subject) was captured and analyzed using the device 200 showing an inhaler technique with variable inspiratory flow rate. Subsequently, individualized feedback using visual cues to the child was made and an improved breathing pattern 828 (Graph B in FIG. 8G) corresponding closely to the optimal respiratory technique as shown in FIG. 7 was captured using the device 200.

In FIG. 8H, the data is presented from a 13 year old child using the device 200 with the pMDI 302 and VHC 304. The breathing pattern 830 (Graph A in FIG. 8H) of the child (i.e. subject) was captured and analyzed using the device 200 showing an inhalation technique with satisfactory inspiratory flow. Subsequently, individualized feedback to the child was made and an improved breathing pattern 832 (Graph B in FIG. 8H) corresponding closely to the optimal respiratory technique as shown in FIG. 7 was captured using the device 200.

In FIG. 8I, the data is presented from a 8 year old child using the device 200 with the pMDI 302 and VHC 304. The breathing pattern 834 (Graph A in FIG. 8I) of the child (i.e. subject) was captured and analyzed using the device 200 showing an inhaler technique with variable inspiratory flow rate. Subsequently, individualized feedback using visual cues to the child was made and an improved breathing pattern 836 (Graph B in FIG. 8I) corresponding closely to the optimal respiratory technique as shown in FIG. 7 was captured using the device 200.

In FIG. 8J, the data is presented from a 8 year old child using the device 200 with the pMDI 302 and VHC 304. The breathing pattern 838 (Graph A in FIG. 8J) of the child (i.e. subject) was captured and analyzed using the device 200 showing fast breathing with insufficient inspiratory flow rate. Subsequently, individualized feedback using visual cues to the child was made and an improved breathing pattern 840 (Graph B in FIG. 8J) corresponding closely to the optimal respiratory technique as shown in FIG. 7 was captured using the device 200.

From the graphs in the study, common errors in inhaler technique can be identified using the characteristic flow patterns generated by the device 200. Examples of common errors include improper panting breathing pattern, variable and insufficient inspiratory flow rates; having the VHC mouthpiece in the subject's mouth while the subject inhales and exhales through his/her nose; and having the VHC mouthpiece in the subject's mouth while the subject inhales through his/her nose and exhaling through his/her mouth. The characteristics of the air flow pattern obtained by the device 200 may provide detailed information on the subject's inhaler technique (e.g. inspiratory flow rate, breathing rate, breath holding pause, pause between breathes, average time taken for inhalation etc.). This information can be effectively used to provide individualised feedback to the subject to correct their inhaler technique. Such feedback using visual cues has been shown in the study to improve the subject's inhaler technique.

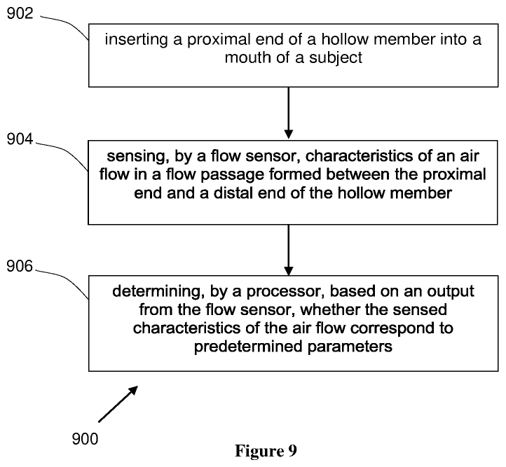

FIG. 9 shows a flow chart illustrating a method for measuring respiratory air flow rate, according to the example embodiments. The method comprises, at step 902, inserting a proximal end of a hollow member into a mouth of a subject. At step 904, the method includes sensing, by a flow sensor, characteristics of an air flow in a flow passage formed between the proximal end and a distal end of the hollow member. At step 904, the method includes determining, by a processor, based on an output from the flow sensor, whether the sensed characteristics of the air flow correspond to predetermined parameters.

The method may further include determining whether the sensed quantity is within a predetermined range; determining whether the sensed characteristics of the air flow correspond to predetermined parameters comprises comparing the sensed flow patterns with stored flow patterns; comparing the sensed PEFR and PIFR with stored PEFR and PIFR and comparing the sensed MEFR and MIFR with stored MEFR and MIFR.

The device and method for measuring respiratory flow as described herein may result in capturing data on adherence with medication (pMDI with VHC device) and inhaler technique in patients (children and adults) with respiratory diseases, accurately and objectively. Embodiments of the invention may address the pitfalls of the existing adherence monitoring methods and may be easy to use, convenient, safe and well tolerated in the clinical setting.

The device and method as disclosed may be capable of objectively and accurately capturing data on adherence with inhaler (or Pressurised Metered Dose Inhaler pMDI) and VHC (or spacer) in patients (children and adults) with respiratory diseases. The device and method may also accurately capture data on inhaler technique by analysis of the flow patterns. The objective assessment of inhaler technique can be used to identify errors in inhaler technique and provide an immediate visual feedback to the patient so that they can correct their technique. The air flow patterns generated by the patient will be useful for the clinician in analysing patient's inhaler technique and providing focused, targeted and individualized inhaler technique education, using visual cues. The device may be a modular unit such that it can be used with the any commercially available existing VHC (or spacer).

The device and method as disclosed can also be used to obtain objective data on the patient's medication adherence during clinic (respiratory/asthma/COPD) reviews. The variabilities in breathing patterns when using the pMDI and the VHC can also be assessed using the device of the present invention.

Embodiments of the invention may provide objective monitoring of treatment adherence in patients with respiratory diseases such as asthma. This would enable clinicians to make informed individualized decisions on asthma management aimed at optimizing patient's asthma control. This may also translate to an improved asthma related quality of life; and a reduction in asthma symptoms, asthma exacerbations, asthma related unscheduled physician/hospital visits, hospital admissions, overall morbidity and cost of care.

Embodiments of the present invention may also analyse the air flow patterns generated by the patient when using with the pMDI with the VHC in real-time. This may identify errors in inhaler technique and provide an immediate visual feedback to the patient so that they can correct their technique.

In addition, embodiments of the present invention may also provide a potential for care transformation. The stored data that is sent to the end user (clinician/specialist nurse) remotely using web based or mobile applications may be helpful in tele-monitoring or virtual clinics as part of restructuring care pathways that involve application of telemedicine. For example, web based tools or mobile applications for assessment of asthma control (that includes symptom review, asthma control test, exacerbation history etc.) may be combined with medication adherence data generated with this invention, for remote monitoring of patients with asthma. Accordingly, appropriate treatment recommendations may be made based on such assessments, thus substantially reducing or minimising the need for face to face clinic/hospital visits. Such tools may also allow better use of scarce resources, targeting patients who need clinic visit for asthma review, while avoiding unnecessary ‘routine’ clinic/hospital follow up visits, for those with well controlled asthma. For the patients, this may translate to reduced number of clinic visits and associated time/cost savings and improved empowerment for managing their asthma/respiratory disease.

Embodiments of the present invention may also provide a potential use in clinical research. This may be achieved through objective assessment of treatment adherence. This is crucial in clinical research when assessing the effect of asthma medications, and hence would be a critical step in asthma drug trials that involves use of pMDls.

It will be appreciated by a person skilled in the art that numerous variations and/or modifications may be made to the present invention as shown in the specific embodiments without departing from the scope of the invention as broadly described. The present embodiments are, therefore, to be considered in all respects to be illustrative and not restrictive.

1. A device for measuring respiratory air flow of a subject, the device comprising:
a hollow member having a proximal end, a distal end and a flow passage formed between the proximal and distal ends, wherein the proximal end is configured to be received in a mouth of a subject;
a flow sensor disposed in the flow passage and configured to sense characteristics of an air flow in the flow passage; and
a processor communicatively coupled to the flow sensor and configured to determine, based on an output from the flow sensor, whether the sensed characteristics of the air flow correspond to predetermined parameters.

2. The device of claim 1, wherein the flow sensor comprises a thermal mass flow sensor, and wherein the processor is configured to translate a temperature output from the thermal mass flow sensor into a flow rate.

3. The device of claim 1, wherein the sensed characteristics of the air flow comprise flow patterns, and wherein the processor is configured to compare the sensed flow patterns with stored flow patterns.

4. The device of claim 1, wherein the sensed characteristics of the air flow comprise peak expiratory flow rate (PEFR) and peak inspiratory flow rate (PIFR), and wherein the processor is configured to compare the sensed PEFR and PIFR with stored PEFR and PIFR data.

5. The device of claim 1, wherein the sensed characteristics of the air flow comprise maximum expiratory flow rate (MEFR) and maximum inspiratory flow rate (MIFR), and wherein the processor is configured to compare the sensed MEFR and MIFR with stored MEFR and MIFR data.

6. The device of claim 1, wherein the device further comprises an indicator configured to display an indication whether the sensed characteristics of the air flow correspond to predetermined parameters.

7. (canceled)

8. (canceled)

9. The device of claim 1, wherein the device further comprises a transmission module communicatively coupled to the processor and configured to transmit the output from the flow sensor to a remote device.

10. The device of claim 2, wherein the distal end of the hollow member is configured to be attached directly to a mouth piece of an inhaler or to a valved holding chamber connected to an inhaler.

11. (canceled)

12. The device of claim 10, wherein the processor is further configured to determine, based on the flow rate, whether the subject inhales a medication dispensed from the inhaler.

13. A method for measuring respiratory air flow of a subject, the method comprising the steps of:
inserting a proximal end of a hollow member into a mouth of the subject;
sensing, by a flow sensor, characteristics of an air flow in a flow passage formed between the proximal end and a distal end of the hollow member; and
determining, by a processor, based on an output from the flow sensor, whether the sensed characteristics of the air flow correspond to predetermined parameters.

14. The method of claim 13, wherein the flow sensor comprises a thermal mass flow sensor, and wherein determining whether the sensed characteristics of the air flow correspond to predetermined parameters comprises translating, by the processor, a temperature output from the thermal mass flow sensor into a flow rate.

15. The method of claim 13, wherein the sensed characteristics of the air flow comprise flow patterns, and wherein determining whether the sensed characteristics of the air flow correspond to predetermined parameters comprises comparing the sensed flow patterns with stored flow patterns.

16. The method of claim 13, wherein the sensed characteristics of the air flow comprise peak expiratory flow rate (PEFR) and peak inspiratory flow rate (PIFR), and wherein the method further comprises comparing the sensed PEFR and PIFR with stored PEFR and PIFR data.

17. The method of claim 13, wherein the sensed characteristics of the air flow comprise maximum expiratory flow rate (MEFR) and maximum inspiratory flow rate (MIFR), and wherein the method further comprises comparing the sensed MEFR and MIFR with stored MEFR and MIFR data.

18. The method of claim 13, further comprising displaying an indication whether the sensed characteristics of the air flow correspond to predetermined parameters in real-time.

19. (canceled)

20. (canceled)

21. The method of claim 13, further comprising transmitting the output from the flow sensor to a remote device.

22. (canceled)

23. The method of claim 14, further comprising attaching the distal end of the hollow member to an inhaler and releasing a medication from the inhaler.

24. The method of claim 23, wherein attaching comprises attaching the distal end of the hollow member directly to a mouthpiece of an inhaler.

25. The method of claim 23, wherein attaching comprises attaching the distal end of the hollow member to one end of a valved holding chamber and attaching the other end of the valved holding chamber to a mouth piece of the inhaler.

26. The method of claim 23, further comprising determining, based on the flow rate, whether the subject inhales a medication dispensed from the inhaler.
